(19) United States
(12) Patent Application Publication
(10) Pub. No.: US 2012/0057125 A1
(43) Pub. Date: Mar. 8, 2012

(54) Adaptive optics ocular fundus observation device

(76) Inventor: Tomohiro Tsuta, Kobe (JP)

(21) Appl. No.: 13/317,327

(22) Filed: Oct. 17, 2011

(30) Foreign Application Priority Data
Nov. 4, 2010 (JP) 2010-260569

(51) Int. Cl. A61B 3/12

(52) U.S. Cl. 351/205

(57) ABSTRACT

An adaptive optics ocular fundus observation device that utilizes adaptive optics in observing ocular fundus, which is characterized by further comprising a judging means for judging whether an arrangement of the retinal locations devoid of nerve cells reflects a retinal nerve fiber tract.

### BACKGROUND OF THE INVENTION

The present invention relates to a device that utilizes adaptive optics in observing ocular fundus.

There is no device that utilizes adaptive optics in observing ocular fundus and further includes a means of the present invention.
Nonpatent literature 1: “The latest comprehensive dictionary of medical science”, Ishiyaku Publishers Inc., 1987, 1990.

The aim of the present invention is to provide an adaptive optics ocular fundus observation device that utilizes adaptive optics in observing ocular fundus, which is characterized by further comprising a judging means for judging whether an arrangement of the retinal locations devoid of nerve cells reflects a retinal nerve fiber tract.

Another aim of the present invention is to provide said adaptive optics ocular fundus observation device, further comprising a highlighting means for highlighting said arrangement on an output device when said arrangement is judged, via said judging means, to reflect the retinal nerve fiber tract.

### SUMMARY OF THE INVENTION

To achieve the above aim, the invention of claim 1 is an adaptive optics ocular fundus observation device that utilizes adaptive optics in observing ocular fundus, which is characterized by further including a judging means for judging whether an arrangement of the retinal locations devoid of nerve cells reflects a retinal nerve fiber tract.

The invention of claim 2 is the adaptive optics ocular fundus observation device of claim 1, further including a highlighting means for highlighting said arrangement on an output device when said arrangement is judged, via said judging means, to reflect the retinal nerve fiber tract.

The invention of claim 3 is a method for an adaptive optics ocular fundus observation device that utilizes adaptive optics in observing ocular fundus, which is characterized by further including a judging step of judging whether an arrangement of the retinal locations devoid of nerve cells reflects a retinal nerve fiber tract.

The invention of claim 4 is the method for the adaptive optics ocular fundus observation device of claim 3, further including a highlighting step of highlighting said arrangement on an output device when said arrangement is judged, via said judging step, to reflect the retinal nerve fiber tract.

According to the invention of claim 1, an arrangement of the retinal locations devoid of nerve cells could be judged to reflect a retinal nerve fiber tract, since the adaptive optics ocular fundus observation device is characterized by further including a judging means for judging whether an arrangement of the retinal locations devoid of nerve cells reflects a retinal nerve fiber tract.

According to the invention of claim 2, said arrangement could be highlighted on an output device when said arrangement is judged, via said judging means, to reflect the retinal nerve fiber tract, since the adaptive optics ocular fundus observation device is characterized by further including a highlighting means for highlighting said arrangement on an output device when said arrangement is judged, via said judging means, to reflect the retinal nerve fiber tract.

### BRIEF DESCRIPTION OF THE DRAWINGS

FIG. 1 is a diagram showing a preferred embodiment of the present invention.

FIG. 2 is a diagram showing a preferred embodiment of the present invention.

FIG. 3 is a diagram showing a preferred embodiment of the present invention.

FIG. 4 is a diagram showing a preferred embodiment of the present invention.

FIG. 5 is a diagram showing a preferred embodiment of the present invention.

### DETAILED DESCRIPTION OF THE INVENTION

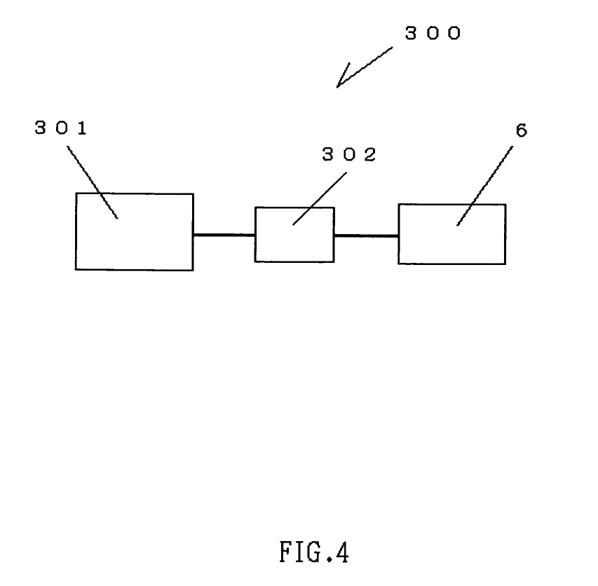

(FIG. 4 shows an adaptive optics ocular fundus observation device system 300 of an adaptive optics ocular fundus observation device, diagrammatically.)

(The present invention of an adaptive optics ocular fundus observation device is realized by the adaptive optics ocular fundus observation device system 300 (for example, carrying out a program for realizing the present invention).)

(As shown in FIG. 4, the adaptive optics ocular fundus observation device system 300 realizing an embodiment of the present invention includes an adaptive optics ocular fundus observation unit 301, a CPU unit 302 equipped with a CPU (Central Processing Unit) 501, etc. which will be mentioned later, (if necessary, a keyboard 303,) (if necessary, a mouse 306,) (if necessary a printer 305,) (if necessary, a speaker 307,) a display 6, etc.)

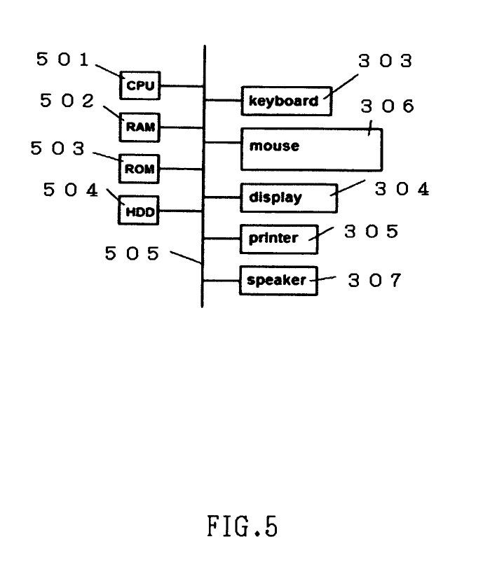

Next, an embodiment of the hardware configuration of the CPU 501 in the present invention is described referring to FIG. 5.

The CPU 501 in the present invention is configured including: a microprocessor such as the CPU 501, a RAM (Random Access Memory) 502, a ROM (Read Only Memory) 503, a HDD (Hard Disc Drive) 504, (a keyboard 303,) (a mouse 306,) a display 304, (a printer 305,) (a speaker 307,) and a communications interface.

These parts are connected via a bus 505, (wireless communication,) etc.

(A CPU unit 302 may (or may not) be incorporated into an adaptive optics ocular fundus observation unit 301.) (for compactness.)

The display 6 is connected through the input-output interface to the bus 505, which enables output to the display 6 of image data input from the CPU 501.

(The printer 305 is connected through the input-output interface to the bus 505, which enables output by the printer 305 of input from the CPU 501.)

The CPU 501 carries out operations characteristic of an embodiment of the present invention (e.g., for the adaptive optics ocular fundus observation unit 301, the CPU unit 302, the display 6, etc.), by loading onto the RAM 502 a program, which is stored in the HDD (Hard Disc Drive) 504, for realizing the present invention.

The CPU 501 carries out controls, and kinds of arithmetic processing, of the present invention, according to a program for realizing the present invention.

The CPU 501 controls display processing of the display 6 (an example of an output device).

(The CPU 501 may control the present invention according to input by the keyboard 303.)
(The CPU 501 may control the printer 305 and the like so as to output results, etc. of the present invention.)
(The keyboard 303 (and if necessary, the mouse 306) and the display 6 may be used as user interfaces in the present invention.)
(The keyboard 303 may be used, for example, as a device for input.)
(If necessary, the mouse 306 may be used as a device for performing various kinds of operations of input to the display screen of the display 6.)

A display 6 may be a display device (an output device), for example, of a LCD (Liquid Crystal Display), a CRT (Cathode Ray Tube), an OLED (Organic Light-Emitting Diode), or the like.

(A display 6 may (or may not) be incorporated into an adaptive optics ocular fundus observation unit 301.) (for compactness.)
(If necessary, various screens such as an operation screen and a setting screen may be displayed on the display 6.)

(And when the CPU 501 is connectable to communications network such as the Internet and a LAN (Local Area Network), the communications interface can be equipped with a network adapter such as a LAN card or communications equipment such as a modem, in order to establish data communication among the network. In such a case, by installing on the network a server storing a program for realizing the present invention, and configuring the CPU 501 as a client terminal of the server, the operation of the present invention may be carried out by an Adaptive optics ocular fundus observation device.)

A program for realizing the present invention may be stored on any computer-readable non-transitory (storage) media (memory device).

Examples of such non-transitory media (storage media) are an optical disk, a magneto-optic disk (CD-ROM, DVD-RAM, DVD-ROM, MO, etc.), a magnetic-storage device (hard disk, Floppy Disk™, ZIP, etc.), a semiconductor memory, etc.

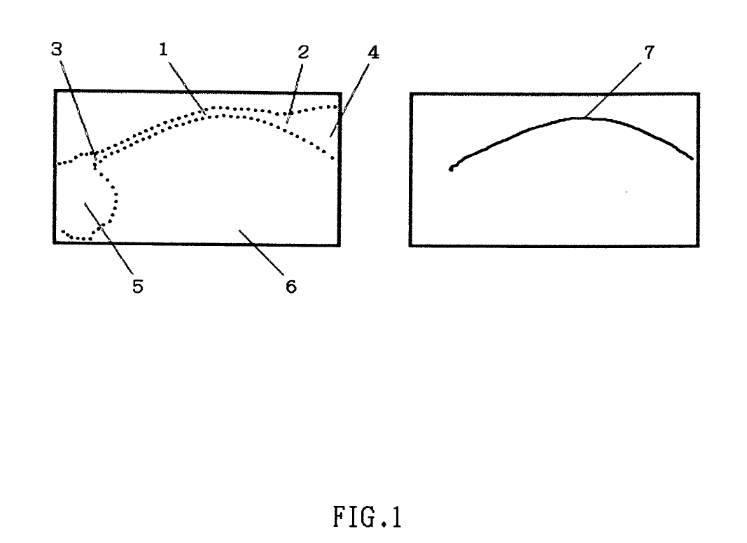

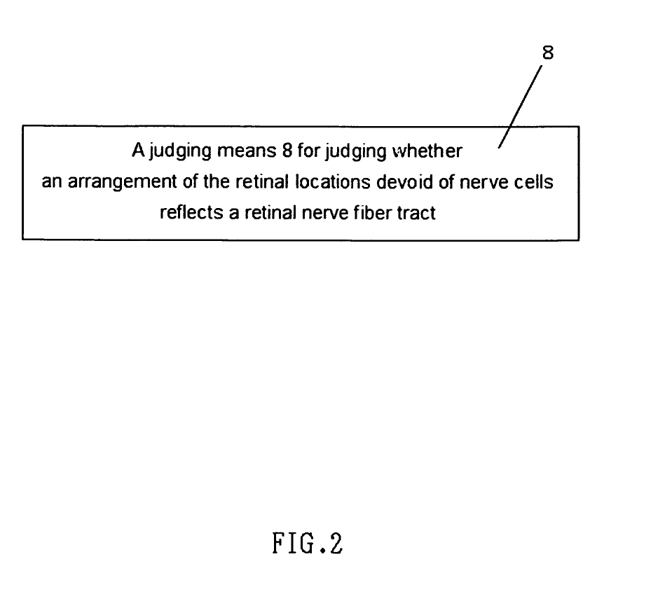

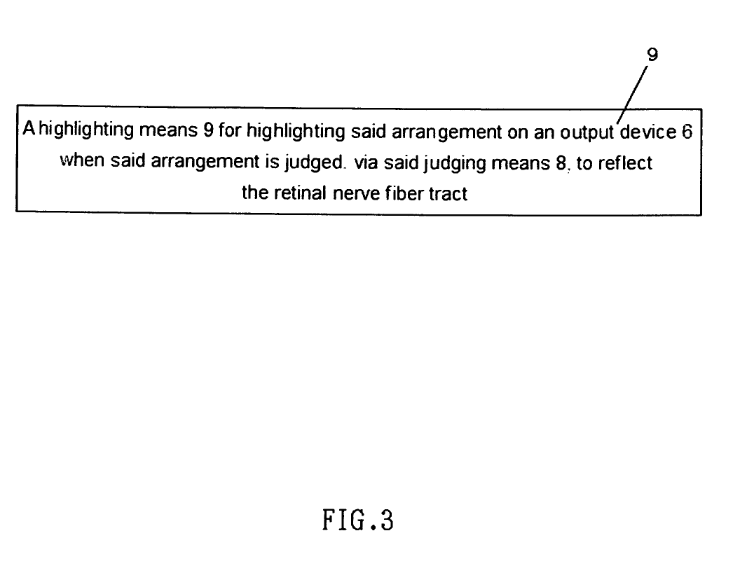

Referring to FIG. 1, FIG. 2, FIG. 3, FIG. 4, and FIG. 5, the present invention will be described.

The present invention of claim 1 is an adaptive optics ocular fundus observation device that utilizes generally known adaptive optics in observing ocular fundus, which is characterized by further including a judging means 8 for judging whether an arrangement of the locations on retina devoid of nerve cells (e.g., an arrangement 1, an arrangement 2, an arrangement 3, etc.) reflects a retinal nerve fiber tract (e.g., a tract 7, etc.).

An arrangement 1 in the present invention of claim 1 refers, for example, to an arrangement of the locations devoid of nerve cells on retina which could be detected between a visual defect region (e.g., a visual defect region 4) and a blind spot (e.g., a blind spot 5) by the adaptive optics ocular fundus observation device in the present invention.

An arrangement 2 in the present invention of claim 1 refers, for example, to an arrangement of the locations devoid of nerve cells on retina which could be detected adjacent to a visual defect region (e.g., a visual defect region 4) by the adaptive optics ocular fundus observation device in the present invention. (It may include the visual defect region (e.g., the visual defect region 4).)

An arrangement 3 in the present invention of claim 1 refers, for example, to an arrangement of the locations devoid of nerve cells on retina which could be detected around and/or adjacent to a blind spot (e.g., a blind spot 5) by the adaptive optics ocular fundus observation device in the present invention.

A device in the present invention of claim 1 that utilizes adaptive optics in observing ocular fundus, for example, refers to a generally known adaptive optics scanning laser ophthalmoscope, a generally known adaptive optics optical coherence tomography, a generally known adaptive optics fundus camera, etc.

(As is generally known, the adaptive optics includes measuring the distortions in a wavefront as generally known, compensating for them using a generally known device such as a deformable mirror, a liquid crystal array, etc.)

A nerve cell (of nerve cells) on retina in the present invention of claim 1 refers, for example, to a photoreceptor cell (a cone cell, a rod cell), a ganglion cell, a bipolar cell, a horizontal cell, an amacrine cell, etc.

The retinal locations devoid of nerve cells in the present invention of claim 1 may include the retinal locations poor in nerve cells which could be detected by the adaptive optics ocular fundus observation device in the present invention.

The retinal locations devoid of nerve cells in the present invention of claim 1 may include the retinal locations with poor reflection (signal) from nerve cells, which could be detected by the adaptive optics ocular fundus observation device in the present invention.

The retinal locations devoid of nerve cells in the present invention of claim 1 may include the retinal (ultra minute) points (each being about (or below) cellular (e.g., a nerve cell) size), each of which shows poor reflection (or poor representation) of a nerve cell, which could be detected by the adaptive optics ocular fundus observation device in the present invention.

An arrangement in the present invention of claim 1 (e.g., an arrangement 1, an arrangement 2, an arrangement 3, etc.) may refer to an arrangement of regions composed of the locations (ultra miniscule points, each being about (or below) cellular (e.g., a nerve cell) size) devoid of nerve cells on retina which could be detected by the adaptive optics ocular fundus observation device in the present invention.

An arrangement in the present invention of claim 1 (e.g., an arrangement 1, an arrangement 2, an arrangement 3, etc.) may refer to an arrangement of the retinal (ultra miniscule points, each being about (or below) cellular (e.g., a nerve cell) size) points, each of which lacks a nerve cell otherwise expected to be there, which could be detected by the adaptive optics ocular fundus observation device in the present invention.

Said locations (points) devoid of nerve cells on retina may be detected (extracted) (in (nearly) cellular resolution) (and stored on a memory device (e.g., the RAM 502, the HDD 504, etc.)) by the adaptive optics ocular fundus observation device of the present invention, for example, via a program (e.g., made of a generally known algorithm such as image processing algorithm, pattern recognition algorithm, image recognition algorithm, etc.) stored on a memory device (e.g., the RAM 502, the HDD 504, etc.) which could enable such detection (for example, based on features (e.g., color, shape, density, reflectance, brightness, size, discontinuity, opaqueness, sharpness, shortness, (distribution of) frequency of the signal reflected off a retinal point, etc. (through being compared, for example, with (proximal) surrounding features (outside the arrangement)) of the retinal locations (points) detected by the adaptive optics ocular fundus observation device of the present invention. (The features may, for example, be local (cellular) features distinct from (proximal) surroundings, or (cellular) features distinct from a normative data (stored on a memory device)).

Said arrangement of the retinal locations devoid of nerve cells may be detected (extracted) (in (nearly) cellular(s) resolution) (and stored on a memory device (e.g., the RAM 502, the HDD 504, etc.)) by the adaptive optics ocular fundus observation device of the present invention, for example, via a program (e.g., made of a generally known algorithm such as image processing algorithm, pattern recognition algorithm, image recognition algorithm, etc.) stored on a memory device (e.g., the RAM 502, the HDD 504, etc.) which could enable such detection (for example, based on features (e.g., color, shape, density, reflectance, brightness, size, discontinuity, opaqueness, sharpness, shortness, (distribution of) frequency of the signal reflected off a retinal point, etc. (through being compared, for example, with (proximal) surrounding features (outside the arrangement)) of the retinal locations (points) detected by the adaptive optics ocular fundus observation device of the present invention. (The features may, for example, be local (cellular(s)) features distinct from (proximal) surroundings, or (cellular) features distinct from a normative data (stored on a memory device)).

In the detection of said arrangement, retinal vascularity (blood vessels of retina) detected by the present invention may be disregarded (or made undistinguished) (for the purpose of detecting the arrangement in relation to (a feature (features) of) nerve cells).

A judging means 8 in the present invention of claim 1 may further include a means for detecting (extracting) an arrangement of regions (poor in nerve cells (in relative comparison with (proximal) surroundings (outside the arrangement))) composed of the locations devoid of nerve cells on retina which could be detected by the adaptive optics ocular fundus observation device of the present invention (e.g., an arrangement 1, an arrangement 2, an arrangement 3, etc.), through generally known image processing of the fundus image obtained from a device that utilizes generally known adaptive optics in observing ocular fundus.

(Such an image processing may be carried out after subdividing the fundus image obtained from the device that utilizes generally known adaptive optics in observing ocular fundus, and averaging pixel intensities, etc. within each subdivided section.)

(Such segmentation may be carried out finer than the diameter of capillary of retina (in order to facilitate the detection of a fine arrangement with the diameter less than that of capillary).)

(Or the segmentation may be carried out coarser than the diameter of capillary of retina.)
The image processing, for example, refers to a generally known edge processing using derivative, filtering, etc.
A (similar local orientation edges extraction) means for (grouping and) extracting the edges whose orientations are nearly identical locally, may further be included.

A retinal nerve fiber tract (e.g., a tract 7, etc.) in the present invention of claim 1 may refer to a retinal nerve fiber tract of a subject that is detected from the subject by a generally known technique (e.g., a generally known (adaptive optics) scanning laser ophthalmoscope, a generally known (adaptive optics) (confocal) optical coherence tomography, a generally known (adaptive optics) fundus camera, a generally known diffusion MRI, a generally known diffusion tensor imaging, a generally known visual field measurement, etc.) (and stored on a memory device (e.g., the RAM 502, the HDD 504, etc.) of the adaptive optics ocular fundus observation device of the present invention).

A retinal nerve fiber tract (e.g., a tract 7, etc.) in the present invention of claim 1 may refer to a (medically) statistical, normative retinal nerve fiber tract based on the data obtained from a generally known technology (e.g., a generally known (adaptive optics) scanning laser ophthalmoscope, a generally known (adaptive optics) (confocal) optical coherence tomography, a generally known (adaptive optics) fundus camera, a generally known diffusion MRI, a generally known diffusion tensor imaging, a generally known visual field measurement, etc.) (stored on a memory device (e.g., the RAM 502, the HDD 504, etc.) of the adaptive optics ocular fundus observation device of the present invention).

A judgment made by a judging means 8 on whether an arrangement of the locations on retina devoid of nerve cells (e.g., an arrangement 1, an arrangement 2, an arrangement 3, etc.) reflects a retinal nerve fiber tract (e.g., a tract 7, etc.) may be carried out by the adaptive optics ocular fundus observation device of the present invention, for example, via a program (e.g., made of a generally known pattern matching algorithm, a generally known image processing algorithm, a generally known image recognition algorithm, a generally known pattern recognition algorithm, etc.) stored on a memory device (e.g., the RAM 502, the HDD 504, etc.) which enables such judgment (for example, (via a generally known pattern matching, etc.) based on features (e.g., shape, orientation, position, gradient, configuration, etc.) of said arrangement detected (stored) by the adaptive optics ocular fundus observation device of the present invention, and (those of) said retinal nerve fiber tract).

The judging means 8 in the present invention of claim 1 may further include a glaucoma disposition judging means for (objectively) judging the degree of glaucomatous tendency based on whether the arrangement (e.g., an arrangement 1, an arrangement 2, an arrangement 3, etc.) reflects a retinal nerve fiber tract (e.g., a tract 7, etc.).

In this instance, the glaucoma disposition (glaucomatous tendency) may, for example, be the glaucoma disposition (glaucomatous tendency) for a visual defect region 4, and/or the glaucoma disposition (glaucomatous tendency) for an arrangement 1, and/or the glaucoma disposition (glaucomatous tendency) for an arrangement 2, and/or the glaucoma disposition (glaucomatous tendency) for an arrangement 3, and so forth.

(Visual disposition other than glaucoma may also be (objectively) judged based on whether the arrangement (e.g., an arrangement 1, an arrangement 2, an arrangement 3, etc.) reflects a retinal nerve fiber tract (e.g., a tract 7, etc.) (or another (normative) pattern specific to the other visual disposition (stored on a memory device)).)

The judging means 8 in the present invention of claim 1 may further include a continuity judging means for judging the degree of continuousness of the arrangement (e.g., an arrangement 1, an arrangement 2, an arrangement 3, etc.) with respect to along the retinal nerve fiber tract (e.g., a tract 7, etc.). (for example, via a program (e.g., made of a generally known algorithm such as image processing algorithm, pattern recognition algorithm, image recognition algorithm, etc.) stored on a memory device (e.g., the RAM 502, the HDD 504, etc.) which could enable such judgement (for example, based on features (e.g., color, shape, density, reflectance, brightness, size, discontinuity, opaqueness, sharpness, shortness, (distribution of) frequency of the signal reflected off a retinal point, etc. (through being compared, for example, with (proximal) surrounding features (outside the arrangement)) of the retinal locations (points) detected by the adaptive optics ocular fundus observation device of the present invention C the features may, for example, be local (cellular) features distinct from (proximal) surroundings, or (cellular) features distinct from a normative data (stored on a memory device)).)

The judging means 8 in the present invention of claim 1 may further include a similarity judging means for judging the degree of the similarity between the shape of the arrangement (e.g., an arrangement 1, an arrangement 2, an arrangement 3, etc.) and the shape of the retinal nerve fiber tract (e.g., a tract 7, etc.). (for example, via a program (e.g., made of a generally known pattern matching algorithm, a generally known image processing algorithm, a generally known image recognition algorithm, a generally known pattern recognition algorithm, etc.) stored on a memory device (e.g., the RAM 502, the HDD 504, etc.) which enables such judgment (for example, (via a generally known pattern matching, etc.) based on features (e.g., shape, orientation, position, gradient, configuration, etc.) of said arrangement detected (stored) by the adaptive optics ocular fundus observation device of the present invention, and (those of) said retinal nerve fiber tract).)

The present invention of claim 2 is the adaptive optics ocular fundus observation device of claim 1, further including a highlighting means 9 for highlighting said arrangement (e.g., an arrangement 1, an arrangement 2, an arrangement 3, etc.) on an output device 6 when said arrangement (e.g., an arrangement 1, an arrangement 2, an arrangement 3, etc.) is judged, via said judging means 8, to reflect the retinal nerve fiber tract (e.g., a tract 7, etc.).

(For example, in order to allow for displaying (highlighting) said arrangement (e.g., using an intrinsic pixel of the display (for the virtual substitute (to suggest the arrangement)) (at the corresponding position)) on an output device 6 even when said arrangement is a super fine arrangement (extremely miniscule arrangement (of almost cellular(s) size (in width perpendicular to its orientation))) that cannot be resolved (displayed) by (ordinary) display's intrinsic resolution (pixel).)

The highlighting means 9 in the present invention of claim 2 may be a highlighting means for highlighting, on the output device 6 of the adaptive optics ocular fundus observation device of the present invention, said arrangement (e.g., an arrangement 1, an arrangement 2, an arrangement 3, etc.) in a representation suggesting said retinal nerve fiber tract (e.g., a tract 7, etc.), when said arrangement (e.g., an arrangement 1, an arrangement 2, an arrangement 3, etc.) is judged, via said judging means 8, to reflect the retinal nerve fiber tract (e.g., a tract 7, etc.).

What is claimed is:

1. An adaptive optics ocular fundus observation device that utilizes adaptive optics in observing ocular fundus, which is characterized by further comprising a judging means for judging whether an arrangement of the retinal locations devoid of nerve cells reflects a retinal nerve fiber tract.

2. The adaptive optics ocular fundus observation device of claim 1, further comprising a highlighting means for highlighting said arrangement on an output device when said arrangement is judged, via said judging means, to reflect the retinal nerve fiber tract.

3. A method for an adaptive optics ocular fundus observation device that utilizes adaptive optics in observing ocular fundus, which is characterized by further comprising a judging step of judging whether an arrangement of the retinal locations devoid of nerve cells reflects a retinal nerve fiber tract.

4. The method for the adaptive optics ocular fundus observation device of claim 3, further comprising a highlighting step of highlighting said arrangement on an output device when said arrangement is judged, via said judging step, to reflect the retinal nerve fiber tract.
